(19) United States
(12) Patent Application Publication
(10) Pub. No.: US 2023/0212229 A1
(43) Pub. Date: Jul. 6, 2023

(54) PREPARATION METHOD AND APPLICATION OF INTERFERING PEPTIDE TARGETING SARS-CoV-2 N PROTEIN

(71) Applicant: SOOCHOW UNIVERSITY, Suzhou, Jiangsu (CN)

(72) Inventors: Fangfang ZHOU, Suzhou (CN); Tong DAI, Suzhou (CN); Shuai WANG, Suzhou (CN); Long ZHANG, Suzhou (CN)

(73) Assignee: SOOCHOW UNIVERSITY, Suzhou, Jiangsu (CN)

(21) Appl. No.: 17/920,637

(22) PCT Filed: Jan. 11, 2022
(86) PCT No.: PCT/CN2022/071356

§ 371 Date: Oct. 21, 2022

(30) Foreign Application Priority Data
Apr. 28, 2021 (CN) 202110466103.7

(51) Int. Cl. C07K 14/005; C07K 14/00; A61P 31/14; A61P 11/00

(52) U.S. Cl. CPC C07K 14/005; C07K 14/001; A61P 31/14; A61P 11/00; C12N 2770/20022; C12N 2740/16022; C12N 2770/20071; C12N 2740/16071; A61K 38/00

(57) ABSTRACT

A preparation method of an interfering peptide targeting SARS-CoV-2 N protein includes the following steps: designing an interfering peptide segment targeting amino acids located in a dimerization domain of the SARS-CoV-2 N protein; fusing the interfering peptide segment with HIV-TAT; modifying the interfering peptide segment fused with HIV-TAT into a reverse isomer to obtain an amino acid sequence of a final interfering peptide NIP-V; and synthesizing the interfering peptide NIP-V using D-amino acids as raw materials. The above-mentioned interfering peptide drug NIP-V is able to interact with the dimerization domain of the SARS-CoV-2 N protein, inhibit the oligomerization of N protein, and then relieve the inhibition for innate immunity by the N protein, so as to achieve the purpose of inhibiting the replication of SARS-CoV-2 virus in cells and animals.

### TECHNICAL FIELD

The present invention belongs to the technical field of drug preparation, in particular to a preparation method and an application of an interfering peptide targeting SARS-CoV-2 N protein.

### BACKGROUND ART

SARS-CoV-2 belongs to the genus Coronavirus (CoV), and its basic structure is composed of spike (S) protein, envelope (E) protein, membrane (M) protein, nucleocapsid (N) protein, and genomic single-stranded RNA. The N protein is the core component of the virus particle. The full-length SARS-CoV-2 N protein includes 419 amino acids, and is mainly composed of an N-terminal RNA-binding domain, a C-terminal dimerization domain, and other sequences connecting these two domains. The full sequence of the SARS-CoV-2 N protein contains a number of relatively conserved regions of positive charge distribution that can bind to RNA, which can bind to viral genomic RNA and then package the RNA into a ribonucleocapsid (RNP) complex. In addition, the dimerization domain can mediate the formation of homo-oligomers of the SARS-CoV-2 N protein. At present, in the studies related to the prevention and treatment of new coronavirus pneumonia, the prevention is dominated by various types of SARS-CoV-2 vaccines which are gradually vaccinated to people around the world. However, there is a lack of specific drugs for treatment. Small-molecule drugs generally have a long development cycle, and currently all of them are existing drugs for new applications. Moreover, the efficacy of these drugs is questioned, such as hydroxychloroquine, lopinavir/ritonavir and remdesivir. In addition to small molecule drugs, plasma therapy is also a good treatment, but there are certain risks, as plasma varies from person to person, it is a complex mixture, and plasma sources are limited and cannot be used on a large scale.

Therefore, it is necessary to propose further solutions to address the problems mentioned above.

### SUMMARY OF THE INVENTION

One object of the present invention is to provide a preparation method of an interfering peptide targeting SARS-CoV-2 N protein, so as to prepare an interfering peptide drug (named as NIP-V hereinafter) for inhibiting the replication and proliferation of SARS-CoV-2 in cells and animals to achieve the treatment of SARS-CoV-2 infection-related diseases.

A technical solution of the present invention is as follows: a preparation method of an interfering peptide targeting the SARS-CoV-2 N protein is provided, the method comprises the following steps:

(a) designing an interfering peptide segment targeting amino acids located in a dimerization domain of the SARS-CoV-2 N protein;

(b) fusing the interfering peptide segment with HIV-TAT;

(c) modifying the interfering peptide segment fused with HIV-TAT into a reverse isomer to obtain an amino acid sequence of a final interfering peptide NIP-V; and

(d) synthesizing the interfering peptide NIP-V using D-amino acids as raw materials.

Further, in step (a), the amino acids are amino acids 346 to 357.

Further, an amino acid sequence of the amino acids 346 to 357 is FKDQVILLNKHI.

Further, the amino acids are L-type natural amino acids.

Further, in step (b), an amino acid sequence of the HIV-TAT is YGRKKRRQRRR.

Further, in step (c), an amino acid sequence of the final interfering peptide NIP-V is IHKNLLIVQDKFPPRRRQRRKKRG, and a molecular weight thereof is 3040.69.

Another technical solution of the present invention is as follows: an application of an interfering peptide targeting SARS-CoV-2 N protein in anti-SARS-CoV-2 infection.

The present invention provides a preparation method and an application of an interfering peptide NIP-V targeting the SARS-CoV-2 N protein, and has the following advantages:

1. The interfering peptide NIP-V targeting the SARS-CoV-2 N protein can effectively relieve the antiviral immunosuppression mediated by the SARS-CoV-2 N protein, and significantly prevent the replication and proliferation of SARS-CoV-2 in transgenic mice expressing human angiotensin-converting enzyme 2 (ACE2), and improve the ability of mice to resist SARS-CoV-2.

2. The interactions between proteins are usually surface-surface interactions; compared with traditional small molecule drugs, the interfering peptide NIP-V targeting the SARS-CoV-2 N protein provided in the present invention can effectively block the self-interaction of the SARS-CoV-2 N protein.

3. The interfering peptide NIP-V targeting the SARS-CoV-2 N protein contains the HIV-TAT sequence, which can directly make the peptide pass through the cell membrane and enter the cytoplasm to play a role without any carrier, thereby avoiding the toxic and side effects that may be caused by a carrier.

4. Compared with natural L-amino acids, D-amino acids degrade more slowly in animals. Modification of interfering peptides to D-retroinverso isomers (DRIs) has been shown to be well tolerated and therapeutically effective in previous clinical trials. Therefore, the DRI-modified interfering peptide NIP-V targeting the SARS-CoV-2 N protein is feasible for clinical trials.

5. The interfering peptide NIP-V targeting the SARS-CoV-2 N protein is only a small peptide of 24 amino acids in length. According to the principle of immunology, it is not immunogenic and can avoid causing hypersensitivity reactions.

6. The interfering peptide NIP-V targeting the SARS-CoV-2 N protein can be obtained directly through the existing mature peptide synthesis technologies, with high purity, controllable quality and great drug potential.

### BRIEF DESCRIPTION OF THE DRAWINGS

The left panel of FIG. 1 is a tertiary structure diagram of a dimerization domain (DD) dimer of SARS-CoV-2 N protein, and the right panel thereof is a target sequence diagram of the interfering peptide NIP-V.

FIG. 2 is a schematic diagram of the molecular weight of a synthesized interfering peptide NIP-V as detected by mass spectrometry (MS).

FIG. 3 is a schematic diagram of the purity of a synthesized interfering peptide NIP-V as detected by high performance liquid chromatography (HPLC).

FIG. 4 is a schematic diagram of nucleic acid detection analysis showing that treatment of ACE2 transgenic mice with the interfering peptide NIP-V can significantly inhibit the proliferation of SARS-CoV-2 in lung tissue.

FIG. 5 is a schematic diagram showing by hematoxylin-eosin (HE) staining that treatment of ACE2 transgenic mice with the interfering peptide NIP-V can inhibit the lung lesions caused by SARS-CoV-2 infection.

FIG. 6 is a schematic diagram showing in immunofluorescence experiments that treatment of ACE2 transgenic mice with the interfering peptide NIP-V can inhibit the expression of SARS-CoV-2 N protein in the lungs after SARS-CoV-2 infection.

FIG. 7 is a schematic diagram showing in immunohistochemical experiments that treatment of ACE2 transgenic mice with the interfering peptide NIP-V can inhibit the expression of SARS-CoV-2 S protein in the lungs after SARS-CoV-2 infection.

FIG. 8 is a schematic diagram showing that the interfering peptide NIP-V can enhance the secretion of IFN-β in the serum of ACE2 transgenic mice infected with SARS-CoV-2.

FIG. 9 is a schematic diagram showing that the interfering peptide NIP-V can enhance the expression of mRNAs of IFN-β and ISG56 in spleen, liver and lung tissue of SARS-CoV-2 infected ACE2 transgenic mice, and reduce the load of SARS-CoV-2 genomic RNA.

FIG. 10 is a schematic diagram showing that the interfering peptide NIP-V can relieve the inhibition of SARS-CoV-2 N protein on the oligomerization of MAVS, a key adaptor protein of the innate immune signaling pathway.

### DESCRIPTION OF THE EMBODIMENTS

A preparation method of an interfering peptide targeting the SARS-CoV-2 N protein is provided in the present invention. Due to the large interaction area of protein-protein interactions, a single small molecule may not be able to interfere effectively; while it can be very effective to use macromolecular drugs such as peptides with similar functional surfaces (interfering peptides) to interfere with protein-protein interactions. Based on the basic function of the SARS-CoV-2 N protein and the mechanism of inhibiting the body's innate antiviral immunity, an interfering peptide targeting N protein has been artificially designed and then obtained by synthesis. This interfering peptide is able to disrupt the hydrophobic interaction by binding to the dimerization domain that mediates the oligomerization of the SARS-CoV-2 N protein, so as to release the N protein's inhibition on the innate immunity and achieve the purpose of inhibiting the replication of SARS-CoV-2 virus in cells, thereby achieving effective treatment of certain clinical conditions.

In the preparation method of an interfering peptide targeting SARS-CoV-2 N protein according to the present invention, firstly, an interfering peptide is designed to target the amino acids 346 to 357 in the N protein dimerization domain of SARS-CoV-2, where the amino acid sequence of the foreoging segment is FKDQVILLNKHI. Natural amino acids are L-form amino acids. This short peptide is designed to specifically disrupt the interaction between N proteins.

Secondly, in order to promote the uptake of NIP-V by cells, the interfering peptide is further designed to be fused to HIV-TAT. HIV-TAT is a hydrophilic sequence that has an amino acid sequence of YGRKKRRQRRR. It can enable a peptide to cross the cell membrane in an energy-independent manner to be absorbed by the cell.

Next, as shown in in vivo assays, a DRI-modified peptide can improve the stability and effectiveness of the peptide in cells and animals. Thus, the entire interfering peptide segment is then modified into a reverse isomer, and the final amino acid sequence of the interfering peptide NIP-V is as follows: IHKNLLIVQDKFPPRRRQRRKKRG, and the molecular weight thereof is 3040.69.

Finally, the interfering peptide NIP-V is obtained by synthesizing using D-amino acids as raw materials, and the purity of the peptide can be higher than 98%.

In order to make the above objects, features and advantages of the present invention easy to understand, the technical solutions of the present invention will be further described below with reference to the embodiments. However, the present invention is not limited to the embodiments described below, but should include any known modifications within the scope of protection of the present invention as claimed.

Reference herein to “one embodiment” or “an embodiment” refers to a particular feature, structure, or characteristic that may be included in at least one implementation of the present invention. The appearances of “in one embodiment” in various places in this description may not all refer to the same embodiment, nor are they separate or selectively mutually exclusive from other embodiments.

### Embodiment 1

Synthesis and Detection of Interfering Peptide Drug NIP-V

The amino acid sequence of the interfering peptide drug NIP-V designed by the present invention is IHKNLLIVQDKFPPRRRQRRKKRG. The targeting sequence is as shown in FIG. 1. It has been synthesized by GL Biochemical (Shanghai) Co., Ltd. using D-amino acids as the raw material.

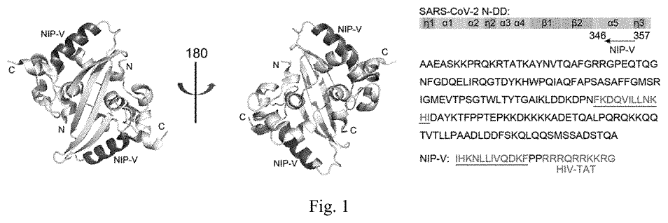

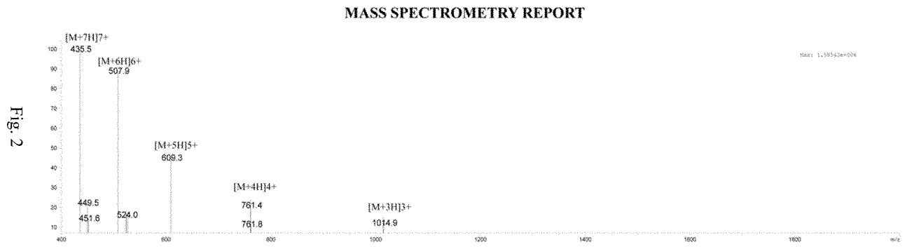

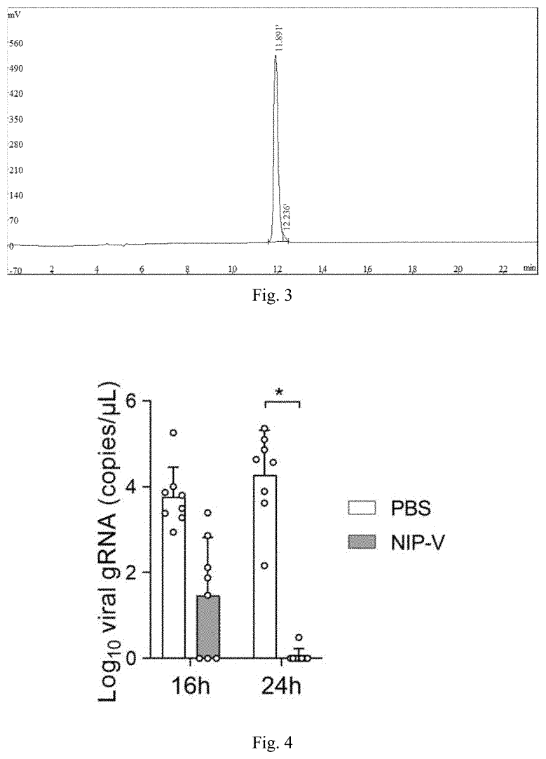

As shown in FIG. 2, the synthesized interfering peptide drug NIP-V is identified by the Agilent-6125B LC/MS system (Agilent Technologies) to have a molecular weight of 3040.69. The HPLC experiment uses the Inertsil ODS-SP liquid chromatography column (Shimadzu, 4.6 mm×250 mm) as the stationary phase, along with the mobile phase A (100% acetonitrile, 0.1% trifluoroacetic acid) and the mobile phase B (100% ultrapure water, 0.1% trifluoroacetic acid) for gradient elution. As shown in FIG. 3 and Table 1, the purity of the product is greater than 98% as determined by HPLC.

TABLE 1

| Retention time | Content (%) | Peak area | Peak height |
|---|---|---|---|
| 11.891 | 98.03 | 6613214 | 514625 |
| 12.236 | 1.966 | 132591 | 20430 |

### Embodiment 2

Treatment of ACE2 transgenic mice with the interfering peptide drug NIP-V can significantly reduce the proliferation of SARS-CoV-2 in mice.

1 Experimental Materials

ACE2 transgenic mice, DAAN Gene novel coronavirus (2019-nCoV) nucleic acid detection kit (fluorescence PCR method), SARS-CoV-2, the NIP-V interfering peptide drug prepared in Embodiment 1.

2 Experimental Methods

The NIP-V interfering peptide drug is dissolved in sterile PBS to reach a concentration of 1 mg/mL. ACE2 transgenic mice are divided into 4 groups with 8 mice in each group. The first and third groups are injected with 0.5 mL sterilized PBS as a control; the second and fourth groups are injected with 0.5 mL (0.5 mg) of the NIP-V drug. 1 hour after the foregoing treatment, all four groups of mice are anesthetized and intranasally inoculated with SARS-CoV-2 at approximately 1×10⁵ TCID50 virus per mouse. 16 hours and 24 hours after the infection with the virus, the first and second groups, and the third and fourth groups of mouse lung tissues are taken respectively, and the lung tissues of the mice are then tested by the DAAN Gene novel coronavirus (2019-nCoV) nucleic acid detection kit to determine the nucleic acid content of SARS-CoV-2. Statistical analysis of the results is expressed as “mean±standard deviation” (mean±SEM). Analysis of variance (ANOVA) is used for comparison, p<0.05 is considered as significantly different, and p<0.01 is considered as significantly very different.

3 Experimental Results

Please refer to FIG. 4, as shown in FIG. 4, the interfering peptide drug NIP-V can significantly reduce the load of SARS-CoV-2 in the lung tissue of ACE2 transgenic mice.

Nucleic acid detection kits are one of the commonly used methods to detect the viral load of SARS-CoV-2. The absolute quantitative PCR method is used to detect the SARS-CoV-2 genomic RNA copy number in tissues with the DAAN Gene novel coronavirus (2019-nCoV) nucleic acid detection kit. The results are shown in Table 2 below.

TABLE 2

| Mouse | | | | | | | | | |
|---|---|---|---|---|---|---|---|---|---|
| group | Treatment | SARS-CoV-2 copies/μL, n = 8 | | | | | | | |
| 1 | PBS + SARS-CoV2 16 h | 7491 | 6412 | 183456 | 10136 | 2446 | 1913 | 879 | 3175 |
| 2 | NIP-V + SARS-CoV2 16 h | 30 | 76 | 0 | 0 | 2484 | 131 | 0 | 726 |
| 3 | PBS + SARS-CoV2 24 h | 7756 | 4231 | 36874 | 127523 | 74107 | 231895 | 42961 | 145 |
| 4 | NIP-V + SARS-CoV2 24 h | 3 | 0 | 0 | 0 | 0 | 0 | 0 | 0 |

The results in Table 2 show that in the PBS-treated groups (groups 1 and 3), the SARS-CoV-2 viral load has an upward trend over time; while in the NIP-V-treated groups (groups 2 and 4), the SARS-CoV-2 viral load is greatly reduced, and SARS-CoV-2 cannot be detected in most mouse lung tissues. Hence, it can be seen that the interfering peptide drug NIP-V is able to significantly reduce the load of SARS-CoV-2 in the lung tissue of ACE2 transgenic mice.

### Embodiment 3

Treatment of ACE2 transgenic mice with the interfering peptide drug NIP-V can significantly inhibit SARS-CoV-2 infection-induced lung lesions in mice.

1 Experimental Materials

ACE2 transgenic mice, SARS-CoV-2, the NIP-V interfering peptide drug prepared in Embodiment 1, tissue fixation, embedding and related materials, and reagents for HE staining (Sangon).

2 Experimental Methods

The NIP-V interfering peptide drug is dissolved in sterile PBS to reach a concentration of 1 mg/mL. ACE2 transgenic mice are divided into 3 groups, the first group is not infected; the second group is injected with 0.5 mL of sterilized PBS and then intranasally inoculated with 1×10⁵ TCID50 of SARS-CoV-2 1 hour after the injection; the third group is injected with 0.5 mg of the NIP-V drug and then intranasally inoculated with 1×10⁵ TCID50 of SARS-CoV-2 1 hour after the injection. 24 hours after the viral infection, mouse lung tissues are taken and placed in 4% paraformaldehyde/PBS for tissue fixation, and paraffin sections of lung tissues are prepared. The lesions on mouse lungs are then detected by HE staining.

3 Experimental Results

Please refer to FIG. 5, as shown in FIG. 5, the interfering peptide drug NIP-V can significantly reduce the lung lesions of ACE2 transgenic mice caused by SARS-CoV-2 infection. The lung tissues of the virus-uninfected mice are normal in morphology, with clear alveoli and thin septa. However, in the PBS+SARS-CoV-2 treatment group, the alveolar septum is significantly thickened, and hemagglutination and inflammatory cell infiltration caused by viral infection can be seen locally, indicating that viral infection can cause a significant inflammatory response. The thickening of alveolar septa is not significant in the NIP-V+SARS-CoV-2 treatment group. In addition, the occurrence of hemagglutination and inflammatory cell infiltration in this group is significantly lower than that in the PBS+SARS-CoV-2 group.

### Embodiment 4

Treatment of ACE2 transgenic mice with the interfering peptide drug NIP-V can significantly inhibit the expression of N and S proteins of SARS-CoV-2 in mouse lung tissue.

1 Experimental Materials

ACE2 transgenic mice, SARS-CoV-2, the NIP-V interfering peptide drug prepared in Embodiment 1; the materials and reagents related to tissue fixation and embedding, such as paraformaldehyde, are all domestically produced; rabbit anti-SARS-CoV-2 N protein antibody (Abcam), mouse anti-SARS-CoV-2 S protein antibody (Abcam), DAPI, FITC-conjugated goat anti-rabbit IgG, HRP-conjugated goat anti-mouse IgG (CST), DAB chromogenic kit (Sangon)

2 Experimental Methods

The drug administration and virus stimulation of ACE2 transgenic mice in this study are the same as those described in Embodiment 3. 24 hours after the SARS-CoV-2 infection, mouse lung tissues are taken and fixed in 4% paraformaldehyde/PBS to make paraffin sections of lung tissues. The expression of the SARS-CoV-2 S protein in the lungs of mice is detected by immunohistochemistry. The expression of the SARS-CoV-2 N protein in the lungs of mice is detected by immunofluorescence assay.

3 Experimental Results

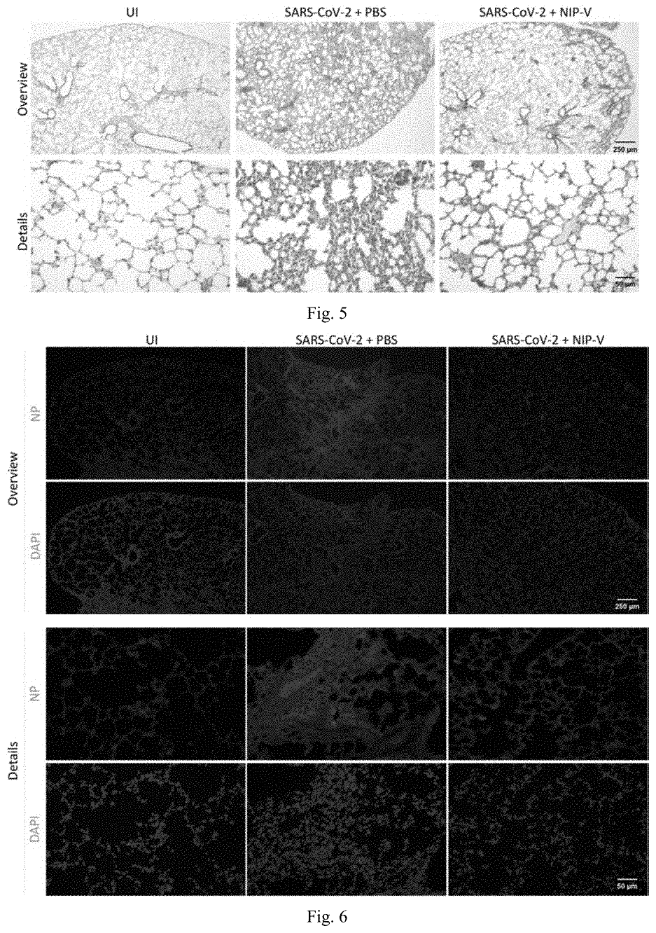

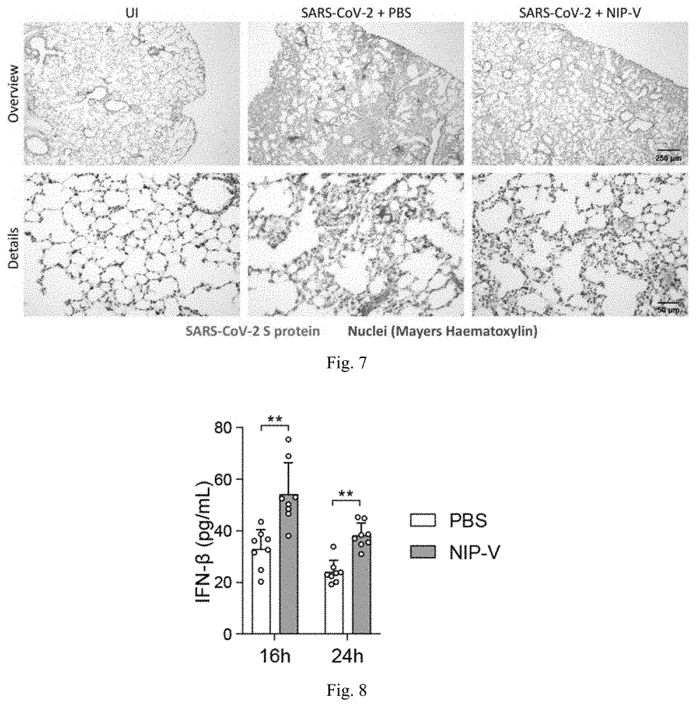

Please refer to FIGS. 6 and 7, as shown in FIG. 6, the expression of SARS-CoV-2 N protein is detected with a FITC-conjugated fluorescent secondary antibody; there is almost no fluorescence signal in the lung tissue of the mice without virus infection; while there is a strong fluorescence signal in the lung tissue of the mice in the PBS+SARS-CoV-2 treatment group; in the lung tissue of the NIP-V+SARS-CoV-2-treated mice, weaker N protein signals can be detected. Similarly, as shown in FIG. 7, the expression of SARS-CoV-2 S protein in the lungs of ACE2 transgenic mice is examined by means of immunohistochemistry. The results show that brown-red staining appears at the site where S protein is present by demonstrated by the DAB chromogenic method. In addition, no S protein signal is shown in the lung tissue of the mice without virus infection; while strong S protein signal and inflammatory cell infiltration are shown in the PBS+SARS-CoV-2 treated mice; yet the S protein signal in the lung tissue of the NIP-V+SARS-CoV-2 treated mice is much lower than that of the PBS+SARS-CoV-2 treated mice. Hence, it can be seen that the interfering peptide drug NIP-V can significantly reduce the expression of SARS-CoV-2 N and S proteins in the lungs of ACE2 transgenic mice infected with SARS-CoV-2.

### Embodiment 5

Treatment of ACE2 transgenic mice with the interfering peptide drug NIP-V can significantly enhance the antiviral innate immune response of the mice infected with SARS-CoV-2 and reduce viral proliferation in tissues.

1 Experimental Materials

ACE2 transgenic mice, SARS-CoV-2, the NIP-V interfering peptide drug prepared in Embodiment 1, mouse IFN-β ELISA detection kit, Trizol Japan (TAKARA), reverse transcription kit, qPCR kit. Table 3 shows the primers required for qPCR (Genewiz)

TABLE 3

| Primers | Sequence (5′-3′) |
|---|---|
| Murine 18S forward | CGCGGTTCTATTTTGTTGGT |
| Murine 18S reverse | AGTCGGCATCGTTTATGGTC |
| Murine Ifnb1 forward | TCCTGCTGTGCTTCTCCACCACA |
| Murine Ifnb1 reverse | AAGTCCGCCCTGTAGGTGAGGTT |
| Murine Isg56 forward | AAGACAAGGCAATCACCCTCTACT |
| Murine Isg56 reverse | GTCTTTCAGCCACTTTCTCCAAA |
| SARS-CoV-2 forward | CTTCTCGTTCCTCATCACGTAGTC |
| SARS-CoV-2 reverse | TTGCTCTCAAGCTGGTTCAATC |

2 Experimental Methods

The drug administration and virus stimulation of ACE2 transgenic mice are the same as those described in Embodiment 1. 16 and 24 hours after the SARS-CoV-2 infection, blood samples are collected from the orbits of mice. Blood cells are then removed by centrifugation at 1200 rpm for 5 min, and serum is retained. The content of IFN-β in serum is detected with the mouse IFN-β ELISA detection kit. The spleen, liver and lung tissues of mice are taken, and total RNA samples are extracted by the Trizol method. After reverse transcription, the expression of Ifnb1 and Isg56 mRNAs in spleen, liver and lung tissues and the load of SARS-CoV-2 genomic RNA are detected by qPCR. Statistical analysis of the results is expressed as “mean±standard deviation” (mean±SEM). Analysis of variance (ANOVA) is used for comparison, p<0.05 is considered as significantly different, and p<0.01 is considered as significantly very different.

3 Experimental Results

The content of IFN-β in serum is detected with the mouse IFN-β ELISA detection kit, and the results are shown in Table 4 below.

TABLE 4

| Mouse | | | | | | | | | |
|---|---|---|---|---|---|---|---|---|---|
| group | Treatment | IFN-β in serum (pg/mL n = 8) | | | | | | | |
| 1 | PBS + SARS-CoV2 16 h | 31.59 | 32.55 | 24.78 | 43.40 | 36.06 | 20.21 | 38.61 | 36.70 |
| 2 | NIP-V + SARS-CoV2 16 h | 52.44 | 46.60 | 50.21 | 75.33 | 37.97 | 52.98 | 68.93 | 48.49 |
| 3 | PBS + SARS-CoV2 24 h | 20.09 | 23.61 | 33.82 | 23.93 | 22.97 | 25.84 | 22.33 | 19.14 |
| 4 | NIP-V + SARS-CoV2 24 h | 38.29 | 38.61 | 34.78 | 30.95 | 44.88 | 37.02 | 35.42 | 45.20 |

As shown in Table 4, the interfering peptide drug NIP-V can significantly increase the content of IFN-β in the serum of ACE2 transgenic mice infected with SARS-CoV-2. Please refer to FIG. 8. As shown in FIG. 8, in the PBS-treated groups (groups 1 and 3), the serous IFN-β in the mice infected with SARS-CoV-2 shows a downward trend with time; while in the NIP-V-treated group (groups 2 and 4), the content of IFN-β in the mice is significantly higher than that in the PBS-treated group (FIG. 8). Hence, it can be seen that the interfering peptide drug NIP-V can significantly increase the IFN-β content in the serum of ACE2 transgenic mice infected with SARS-CoV-2.

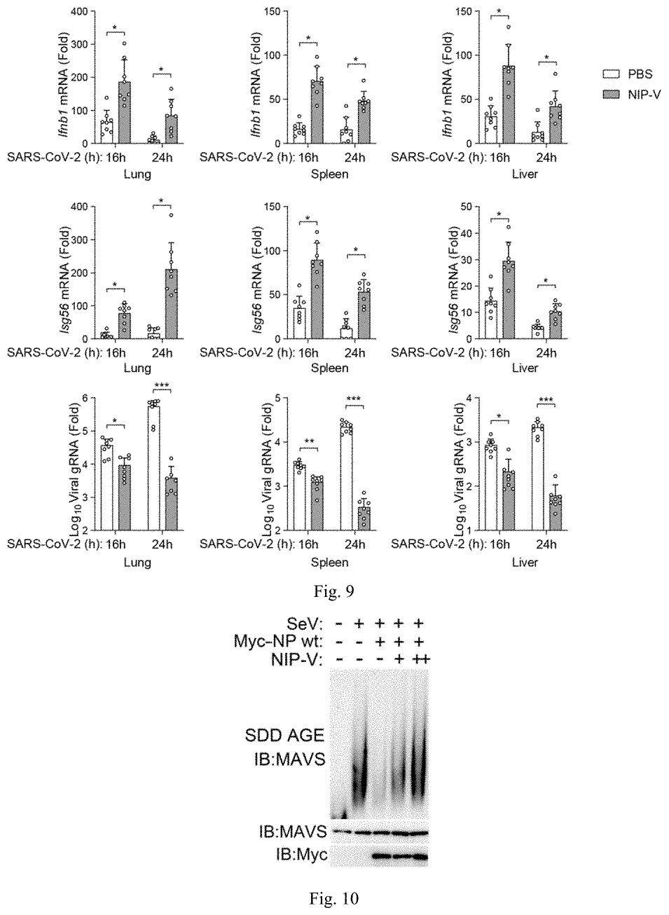

As shown in FIG. 9, after pretreating ACE2 transgenic mice with the interfering peptide drug NIP-V, the expression of Ifnb 1 and Isg56 mRNAs induced by the infection with SARS-CoV-2 is increased, as compared with the PBS-treated group, which indicates that the NIP-V treatment is able to enhance the antiviral innate immune response to SARS-CoV-2 in mice, and reduce the proliferation of SARS-CoV-2 in tissues. The results of the increases in mRNAs relative to the unstimulated mice are shown in Table 5 below.

TABLE 5

| Lung | | Ifnb1 mRNA (Fold, n = 8) | | | | | | | |
|---|---|---|---|---|---|---|---|---|---|
| | PBS + SARS-CoV2 | 138.33 | 70.13 | 62.77 | 42.28 | 34.11 | 27.51 | 83.40 | 65.44 |
| | 16 h | | | | | | | | |
| | NIP-V + SARS-CoV2 | 186.37 | 302.75 | 202.16 | 145.17 | 113.93 | 133.62 | 259.88 | 148.33 |
| | 16 h | | | | | | | | |
| | PBS + SARS-CoV2 | 8.31 | 11.04 | 17.13 | 31.43 | 21.77 | 0.00 | 0.00 | 0.00 |
| | 24 h | | | | | | | | |
| | NIP-V + SARS-CoV2 | 82.49 | 165.42 | 112.99 | 126.62 | 84.22 | 36.61 | 22.54 | 46.19 |
| | 24 h | | | | | | | | |
| | | Isg56 mRNA (Fold, n = 8) | | | | | | | |
| | PBS + SARS-CoV2 | 14.72 | 5.86 | 30.91 | 6.59 | 3.12 | 5.62 | 12.38 | 5.03 |
| | 16 h | | | | | | | | |
| | NIP-V + SARS-CoV2 | 92.65 | 109.88 | 67.64 | 46.59 | 99.03 | 85.62 | 91.13 | 26.35 |
| | 16 h | | | | | | | | |
| | PBS + SARS-CoV2 | 28.89 | 31.78 | 38.32 | 27.47 | 4.50 | 0.00 | 0.00 | 0.00 |
| | 24 h | | | | | | | | |
| | NIP-V + SARS-CoV2 | 232.32 | 263.20 | 152.15 | 131.60 | 144.01 | 188.71 | 205.07 | 374.81 |
| | 24 h | | | | | | | | |
| | | SARS-CoV-2 genomic RNA (Fold, n = 8) | | | | | | | |
| | PBS + SARS-CoV2 | 3.35E+04 | 4.18E+04 | 2.84E+04 | 6.39E+04 | 5.19E+04 | 1.24E+04 | 1.40E+04 | 5.48E+04 |
| | 16 h | | | | | | | | |
| | NIP-V + SARS-CoV2 | 5.93E+03 | 4.43E+03 | 2.73E+03 | 1.61E+04 | 1.86E+04 | 1.48E+04 | 8.10E+03 | 3.62E+03 |
| | 16 h | | | | | | | | |
| | PBS + SARS-CoV2 | 6.78E+05 | 1.35E+05 | 1.09E+05 | 7.31E+05 | 5.39E+05 | 7.95E+05 | 6.45E+05 | 8.11E+05 |
| | 24 h | | | | | | | | |
| | NIP-V + SARS-CoV2 | 4.74E+03 | 3.19E+03 | 3.72E+03 | 1.23E+03 | 1.56E+03 | 1.26E+03 | 1.49E+04 | 0.00E+00 |
| | 24 h | | | | | | | | |
| Liver | | Ifnb1 mRNA (Fold, n = 8) | | | | | | | |
| | PBS + SARS-CoV2 | 34.64 | 15.37 | 27.75 | 23.74 | 33.75 | 41.77 | 50.63 | 18.01 |
| | 16 h | | | | | | | | |
| | NIP-V + SARS-CoV2 | 52.55 | 86.45 | 85.91 | 111.15 | 85.02 | 132.00 | 68.88 | 81.36 |
| | 16 h | | | | | | | | |
| | PBS + SARS-CoV2 | 20.88 | 11.83 | 37.90 | 7.23 | 8.54 | 4.10 | 4.51 | 8.42 |
| | 24 h | | | | | | | | |
| | NIP-V + SARS-CoV2 | 22.59 | 79.02 | 29.53 | 33.69 | 42.93 | 49.66 | 31.43 | 45.92 |
| | 24 h | | | | | | | | |
| | | Isg56 mRNA (Fold, n = 8) | | | | | | | |
| | PBS + SARS-CoV2 | 13.20 | 10.35 | 7.85 | 13.33 | 18.56 | 23.66 | 12.58 | 15.41 |
| | 16 h | | | | | | | | |
| | NIP-V + SARS-CoV2 | 30.49 | 18.13 | 27.16 | 42.29 | 27.58 | 35.86 | 25.80 | 29.09 |
| | 16 h | | | | | | | | |
| | PBS + SARS-CoV2 | 6.72 | 4.40 | 1.86 | 4.14 | 4.22 | 3.97 | 4.53 | 4.08 |
| | 24 h | | | | | | | | |
| | NIP-V + SARS-CoV2 | 14.51 | 10.01 | 7.41 | 10.47 | 12.89 | 12.03 | 9.46 | 5.61 |
| | 24 h | | | | | | | | |
| | | SARS-CoV-2 genomic RNA (Fold, n = 8) | | | | | | | |
| | PBS + SARS-CoV2 | 1.51E+03 | 7.18E+02 | 8.48E+02 | 1.01E+03 | 1.00E+03 | 6.11E+02 | 4.55E+02 | 7.12E+02 |
| | 16 h | | | | | | | | |
| | NIP-V + SARS-CoV2 | 2.09E+02 | 1.42E+02 | 8.55E+01 | 1.08E+02 | 1.69E+02 | 6.80E+02 | 8.67E+01 | 2.38E+02 |
| | 16 h | | | | | | | | |
| | PBS + SARS-CoV2 | 1.32E+03 | 3.35E+03 | 2.85E+03 | 2.32E+03 | 2.35E+03 | 1.85E+03 | 1.11E+03 | 1.96E+03 |
| | 24 h | | | | | | | | |
| | NIP-V + SARS-CoV2 | 6.41E+01 | 4.17E+01 | 4.33E+01 | 1.70E+02 | 5.83E+01 | 5.13E+01 | 3.93E+01 | 2.38E+01 |
| | 24 h | | | | | | | | |
| Spleen | | Ifnb1 mRNA (Fold, n = 8) | | | | | | | |
| | PBS + SARS-CoV2 | 14.80 | 7.99 | 12.27 | 19.94 | 18.99 | 30.86 | 10.91 | 16.65 |
| | 16 h | | | | | | | | |
| | NIP-V + SARS-CoV2 | 87.27 | 42.74 | 73.72 | 58.30 | 67.91 | 98.19 | 64.75 | 70.43 |
| | 16 h | | | | | | | | |
| | PBS + SARS-CoV2 | 2.74 | 17.79 | 29.10 | 10.36 | 0.00 | 11.24 | 13.48 | 42.61 |
| | 24 h | | | | | | | | |
| | NIP-V + SARS-CoV2 | 45.66 | 36.50 | 51.66 | 47.70 | 40.76 | 71.38 | 47.81 | 45.86 |
| | 24 h | | | | | | | | |
| | | Isg56 mRNA (Fold, n = 8) | | | | | | | |
| | PBS + SARS-CoV2 | 18.91 | 41.44 | 22.18 | 61.01 | 29.22 | 25.83 | 36.33 | 41.96 |
| | 16 h | | | | | | | | |
| | NIP-V + SARS-CoV2 | 121.34 | 87.49 | 110.12 | 85.19 | 94.54 | 59.17 | 76.17 | 82.88 |
| | 16 h | | | | | | | | |
| | PBS + SARS-CoV2 | 0.00 | 17.79 | 29.10 | 0.00 | 21.35 | 12.44 | 13.48 | 0.00 |
| | 24 h | | | | | | | | |
| | NIP-V + SARS-CoV2 | 73.25 | 63.36 | 55.41 | 62.68 | 45.16 | 32.90 | 56.97 | 36.50 |
| | 24 h | | | | | | | | |
| | | SARS-CoV-2 genomic RNA (Fold, n = 8) | | | | | | | |
| | PBS + SARS-CoV2 | 2.57E+03 | 4.06E+03 | 2.80E+03 | 3.02E+03 | 3.29E+03 | 2.04E+03 | 2.72E+03 | 3.44E+03 |
| | 16 h | | | | | | | | |
| | NIP-V + SARS-CoV2 | 1.66E+03 | 1.40E+03 | 1.37E+03 | 4.78E+02 | 1.60E+03 | 8.65E+02 | 1.33E+03 | 1.15E+03 |
| | 16 h | | | | | | | | |
| | PBS + SARS-CoV2 | 2.43E+04 | 1.59E+04 | 3.14E+04 | 1.48E+04 | 2.47E+04 | 1.73E+04 | 2.81E+04 | 1.95E+04 |
| | 24 h | | | | | | | | |
| | NIP-V + SARS-CoV2 | 4.18E+02 | 3.11E+02 | 2.38E+02 | 1.85E+02 | 4.43E+02 | 1.35E+02 | 2.56E+02 | 7.20E+02 |
| | 24 h | | | | | | | | |

As shown in Table 5, the qPCR results show that the NIP-V treatment can increase the expression of Ifnb1 and Isg56 mRNAs in the spleen, liver, and lung tissues of SARS-CoV-2-infected ACE2 transgenic mice, and inhibit the proliferation of SARS-CoV-2.

### Embodiment 6

Treatment of cells with the interfering peptide drug NIP-V can relieve the inhibition on MAVS oligomerization by the SARS-CoV-2 N protein.

1 Experimental Materials

SARS-CoV-2 N protein expression plasmid Myc-NP, the NIP-V interfering peptide drug prepared in Embodiment 1, Sendai virus (SeV), fetal bovine serum, DMEM culture medium, penicillin/streptomycin solution (Gibco), HEK 293T cell line (ATCC source), MAVS antibody, Myc antibody and related secondary antibodies (CST)

2 Experimental Methods

HEK 293T cells are plated in a 6-well plate. When the cell density reach 70%, Myc-NP plasmid is used to transfect the cells in 3 wells. After 24 h, cells are treated with 50 μM or 100 μM of NIP-V. 1 h after the treatment, the cells are stimulated with SeV for 8 h. The cells are harvested after 8 h, and the oligomerization of MAVS is then detected by semi-denaturing electrophoresis (SDD-PAGE).

3 Experimental Results

MAVS is an important adaptor protein in the innate immune signaling pathway, and the oligomerization thereof is one of the important signs of the activation of the antiviral innate immune pathway. The SARS-CoV-2 N protein can inhibit the antiviral innate immunity by acting on MAVS. In this study, it is studied whether NIP-V can rescue the innate immune signaling pathway inhibited by the N protein in the SDD-PAGE experiment. As shown in FIG. 10, in the resting state, no oligomerization of MAVS occurrs; SeV stimulation activates the innate immune signaling pathway, which can induce the oligomerization of MAVS; transfection of the N protein in cells can inhibit the SeV-induced MAVS oligomerization; however, pretreatment of cells with NIP-V can restore the MAVS oligomerization in a dose-dependent manner. The foregoing results show that NIP-V can relieve the inhibition of SARS-CoV-2 N protein on MAVS and enhance the innate immune signaling.

In summary, the interfering peptide drug NIP-V provided in the present invention is able to interact with the dimerization domain of the SARS-CoV-2 N protein, inhibit the oligomerization of N protein, and then relieve the inhibition for innate immunity by the N protein, so as to achieve the purpose of inhibiting the replication of SARS-CoV-2 virus in cells and animals.

It should be noted that the above embodiments are only provided to describe the technical solutions of the present invention, but not limit the present invention. Although the present invention has been described in detail with reference to these preferred embodiments, it should be understood by a person skilled in the art that the technical solutions of the present invention can be modified or equivalently replaced without departing from the principles and scope of the technical solutions of the present invention, which should all be included in the scope of protection of the present invention as claimed.

1. A preparation method of an interfering peptide targeting SARS-CoV-2 N protein, comprising the following steps:
(a) designing an interfering peptide segment targeting amino acids located in a dimerization domain of the SARS-CoV-2 N protein;
(b) fusing the interfering peptide segment with HIV-TAT;
(c) modifying the interfering peptide segment fused with HIV-TAT into a reverse isomer to obtain an amino acid sequence of a final interfering peptide NIP-V; and
(d) synthesizing the interfering peptide NIP-V using D-amino acids as raw materials.

2. The preparation method of an interfering peptide targeting SARS-CoV-2 N protein according to claim 1, wherein in step (a), the amino acids are amino acids 346 to 357.

3. The preparation method of an interfering peptide targeting SARS-CoV-2 N protein according to claim 2, wherein an amino acid sequence of the amino acids 346 to 357 is FKDQVILLNKHI.

4. The preparation method of an interfering peptide targeting SARS-CoV-2 N protein according to claim 2, wherein the amino acids are L-type natural amino acids.

5. The preparation method of an interfering peptide targeting SARS-CoV-2 N protein according to claim 1, wherein in step (b), an amino acid sequence of the HIV-TAT is YGRKKRRQRRR.

6. The preparation method of an interfering peptide targeting SARS-CoV-2 N protein according to claim 1, wherein in step (c), an amino acid sequence of the final interfering peptide NIP-V is IHKNLLIVQDKFPPRRRQRRKKRG, and a molecular weight thereof is 3040.69.

7. A method comprising applying an interfering peptide targeting SARS-CoV-2 N protein in anti-SARS-CoV-2 infection.
